(19) United States
(12) Patent Application Publication
(10) Pub. No.: US 2024/0009123 A1
(43) Pub. Date: Jan. 11, 2024

(54) MULTI-SUPPLEMENT DELIVERY SYSTEM

(71) Applicants: CORNELL UNIVERSITY, Ithaca, NY (US); R.C. Bigelow, Inc., Fairfield, CT (US)

(72) Inventors: Alireza Abbaspourrad, Ithaca, NY (US); Michelle Lee, Ithaca, NY (US); Gabe Kis-Horvath, White Plains, NY (US)

(21) Appl. No.: 18/219,431

(22) Filed: Jul. 7, 2023

Related U.S. Application Data
(60) Provisional application No. 63/359,475, filed on Jul. 8, 2022.

(51) Int. Cl. A61K 9/107; A61K 47/36; A61K 31/375; A61K 31/4415; A61K 31/714; A61K 31/355; A61K 31/07; A61K 9/19; A61K 9/00; A61K 9/113; A23L 33/125; A23L 33/15; A23L 33/155; A23L 2/60; A23F 3/16

(52) U.S. Cl. CPC A61K 9/1075; A61K 47/36; A61K 31/375; A61K 31/4415; A61K 31/714; A61K 31/355; A61K 31/07; A61K 9/19; A61K 9/0095; A61K 9/113; A23L 33/125; A23L 33/15; A23L 33/155; A23L 2/60; A23F 3/16

(57) ABSTRACT

A method for preparing a nutritional multi-supplement complex, the method comprising: (i) preparing a dispersion of spheroidal polysaccharide-stabilized complexes comprising a water-soluble polysaccharide and at least one water-soluble nutritional supplement in an aqueous medium, wherein the water-soluble polysaccharide and water-soluble nutritional supplement are complexed with each other by ionic interactions in the spheroidal polysaccharide-stabilized complexes; (ii) preparing an oil-supplement solution comprising at least one oil-soluble nutritional supplement dissolved in a food grade oil; and (iii) blending the dispersion of spheroidal polysaccharide-stabilized complexes with the oil-supplement solution to produce the nutritional multi-supplement complex, wherein the nutritional multi-supplement complex comprises droplets of the oil-supplement solution encapsulated by the spheroidal polysaccharide-stabilized complexes. Also described herein are the produced multi-supplement complex, beverages and foods containing the complex, tea bags containing the complex, and capsules and tablets containing the complex.

### CROSS REFERENCE TO RELATED APPLICATIONS

This application claims the benefit of priority from U.S. Provisional Application No. 63/359,475, filed on Jul. 8, 2022, which is herein incorporated by reference in its entirety.

### FIELD OF THE INVENTION

The present invention generally relates to nutritional multi-supplement complexes for human or animal consumption wherein the complex contains at least one water-soluble nutritional supplement (e.g., vitamin) and at least one oil-soluble nutritional supplement (e.g., vitamin). The present invention more particularly relates to such complexes in which the water- and oil-soluble supplements are stably emulsified together.

### BACKGROUND

Although multi-supplement formulations are well-known, existing formulations are generally not capable of controllable release of both aqueous-soluble and oil-soluble supplements into a beverage, such as a tea. Efforts to produce stable homogeneous aqueous solutions containing both aqueous-soluble and oil-soluble supplements have been largely unsuccessful. Generally, the oil-soluble supplements in a multi-supplement mixture will aggregate and separate from other components when placed in an aqueous environment. The result is an unpalatable beverage along with loss of supplement intake. Sometimes, on mixing or blending, a cloudy (turbid) suspension may be formed, which may also not be desirable. Thus, there would be a significant benefit in a method that could produce a multi-supplement formulation that stably incorporates water- and oil-soluble components into an emulsion that is resistant to aggregation and separation when placed in an aqueous environment, such as a tea.

### SUMMARY

In a first aspect, the present disclosure is directed to a novel method for preparing a nutritional multi-supplement complex that incorporates aqueous-soluble and oil-soluble supplements into a stable arrangement. The stable arrangement can be infused into a beverage, such as a tea, to form a stable homogeneous aqueous solution or dispersion of the stable arrangement with little to no cloudiness or turbidity. The stable arrangement can also be coated with an edible water-soluble coating material (e.g., a polysaccharide, such as maltodextrin) and then spray-dried to produce a solid form of the stable arrangement. The spray-dried form of the stable arrangement can be contacted or mixed with an aqueous solution suitable for human consumption to diffuse both aqueous-soluble and oil-soluble supplements into the aqueous solution, preferably in the substantial or complete absence of cloudiness or turbidity (i.e., resulting in a substantially or completely translucent solution).

The method of preparation includes: (i) preparing an aqueous dispersion of spheroidal polysaccharide-stabilized complexes (SPSCs) comprising a water-soluble polysaccharide and at least one water-soluble nutritional supplement (WSNS) in an aqueous medium, wherein the water-soluble polysaccharide and WSNS in the SPSCs are complexed with each other by ionic interactions; (ii) preparing an oil-supplement solution comprising at least one oil-soluble nutritional supplement (OSNS) dissolved in a food grade oil; and (iii) blending (e.g., by ultrasonication) the dispersion SPSCs with the oil-supplement solution to produce the nutritional multi-supplement complex, wherein the nutritional multi-supplement complex comprises droplets of the oil-supplement solution encapsulated by the SPSCs.

The present disclosure is also directed to a three-step process for preparing the aqueous dispersion of SPSCs in step (i). The three-step process includes: (i-1) preparing a first aqueous solution containing at least one water-soluble nutritional supplement, (i-2) preparing a second aqueous solution containing the water-soluble polysaccharide, and (i-3) mixing the first and second aqueous solutions to form the SPSCs. In some embodiments, the first and second aqueous solutions are adjusted to a pH of 2-8 or a pH of 4-6 or a pH of precisely or about 5. The aqueous dispersion containing the SPSCs also preferably has a pH of 2-8 or a pH of 4-6 or a pH of precisely or about 5.

In another aspect, the present disclosure is directed to a nutritional multi-supplement complex produced by the above process. The nutritional multi-supplement complex includes droplets of an oil-supplement solution encapsulated by the SPSCs, wherein: (a) the oil-supplement solution comprises at least one oil-soluble nutritional supplement (OSNS) dissolved in a food grade oil (e.g., a vegetable oil), and (b) the SPSCs comprise a water-soluble polysaccharide and at least one water-soluble nutritional supplement (WSNS) in an aqueous medium, wherein the water-soluble polysaccharide and WSNS are complexed with each other by ionic interactions in the SPSCs. The nutritional multi-supplement complex can also be in a solid form in which the nutritional multi-supplement is encapsulated by an edible water-soluble coating material, such as a second polysaccharide, such as a dextrin (e.g., maltodextrin) or a gum. The encapsulated multi-supplement complex is typically a solid spray-dried particulate form.

In another aspect, the present disclosure is directed to beverages containing the nutritional multi-supplement complex described above. In the beverage, the nutritional multi-supplement complex is homogeneously dispersed in a base aqueous medium suitable for human consumption. The beverage may be, for example, a flavored beverage or a tea beverage, which may contain one or more components from a tea plant or other botanical source.

In yet another aspect, the present disclosure is directed to a tea bag containing the nutritional multi-supplement complex described above. More specifically, the tea bag contains the nutritional multi-supplement complex described above within a tea bag enclosure, wherein the tea bag may have a pore size cut-off of 50, 100, 200, 250, or 300 microns.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C. FIG. 1A is a schematic representation of the preparation of chitosan-stabilized emulsions for co-delivery of water-soluble and oil-soluble vitamins. FIG. 1B shows surface charges of anthocyanin, vitamin C, vitamin B6, vitamin B12, Vitamin E and Vitamin A. FIG. 1C shows morphologies of the anthocyanin-vitamin-chitosan polyelectrolyte complex.

FIGS. 2A-2C. FIG. 2A is a schematic representation of a spray-drying process. FIG. 2B shows scanning electron microscope (SEM) images of the spray-dried vitamin powders. FIG. 2C is a graph plotting the particle size of the spray-dried vitamin powders.

FIGS. 3A-3D. FIG. 3A shows UV spectra of released vitamins during a tea bag steeping process at 4° C. FIGS. 3B-3D show the release rate of water-soluble vitamins (VC, VB6, and VB12, respectively) as a function of brewing time in water of pH 5 at 4° C.

FIGS. 4A-4C. FIG. 4A shows UV spectra of released vitamins during a tea bag steeping process at 4° C. FIGS. 4B and 4C show release rate of oil-soluble vitamins (VE and VA, respectively) as a function of brewing time in water of pH 5 at 4° C.

FIGS. 5A-5C. FIG. 5A shows UV spectra of released vitamins during a tea bag steeping process at 100° C. FIGS. 5B and 5C show the release rate of oil-soluble vitamins (VE and VA, respectively) as a function of brewing time in water of pH 5 at 100° C.

FIG. 6. Schematic representation of the preparation of W/O/W emulgel emulsions for co-delivery of water-soluble and oil-soluble vitamins.

FIG. 7. Photograph of the tea brewed at 25° C. for 10 minutes with the encapsulated vitamin E in W/O/W emulgel system.

FIGS. 8A-8B. FIG. 8A shows optical microscopy (Left field: showing emulsion particle size stabilized by chitosan; Right field: emulsion stabilized by polyelectrolyte complex). FIG. 8B (top) is a bar graph showing particle size reduction when comparing chitosan-stabilized and complex-stabilized emulsions in the more advanced processing phase. FIG. 8B (bottom) is bar graph showing zeta potential change (mV) between chitosan-stabilized and complex-stabilized emulsions.

FIGS. 9A-9B. FIG. 9A (left) is a visual image showing gradual color change of the complex after adding anthocyanin, vitamin C and chitosan at pH=5. FIG. 9B (right) is a bar graph showing changing surface charges forming polyelectrolyte complexes at pH=5. FIG. 9B (left) is a SEM image of the spray-dried powder, with maltodextrin coating. FIG. 9B (right) is a SEM image of the air-dried particles of anthocyanin plus vitamin C complex.

FIG. 10. Photo on left: Snapshots of perfecting encapsulation efficiency during spray drying: Samples of spray dried containing maltodextrin, with water soluble vitamins (C and B6) and oil soluble vitamins (E and A) obtained with 7.6% loading by weight. Table on right: Calculations between milligrams of powder and the individual vitamins, each of them to target 50% RDA; testing encapsulation efficiency in progress.

FIG. 11. Photographs show comparison between Strategy 1 and Strategy 2. Steeping results in filter tera bag; using different vitamin E, mixed with green tea in each formula: Left: Strategy 1, fabricating double layer W/O/W emulsion with high density internal phase: the encapsulated water dispersible (pre-treated) oil soluble vitamin E resulting turbidity in the final beverage. Right: Strategy 2: emulsion fabrication with the cross-linking, preferred method: Left side of Strategy 2: encapsulating oil-soluble vitamin E (mixed tocopherols, food grade) resulting a clear green tea beverage. Right side of Strategy 2: encapsulated water dispersible (pre-treated) oil soluble vitamin E with green tea in the blend, resulting in turbidity in the ready-to-drink beverage.

### DETAILED DESCRIPTION

In a first aspect, the present disclosure is directed to a method for preparing a nutritional multi-supplement complex containing at least one water-soluble nutritional supplement (WSNS) and at least one oil-soluble nutritional supplement (OSNS). The term “nutritional supplement” (i.e., “supplement”), as used herein, includes any one or more nutrients, including vitamins, pro-vitamins (e.g., beta-carotene), minerals (e.g., potassium, magnesium, calcium, and iron), amino acids, proteins, phytochemicals, anti-oxidants, anthocyanins, fatty acids, oil extracts, enzymes, and co-enzymes. The water-soluble nutritional supplement typically includes at least one water-soluble vitamin. Some examples of water-soluble vitamins include vitamin C and the B vitamins (e.g., one or more of vitamin B1, vitamin B2, vitamin B3, vitamin B5, vitamin B6, vitamin B7, vitamin B12, and folate) and active derivatives thereof. The vitamin B12 may be, for example, cyanocobalamin, adenosylcobalamin, methylcobalamin, or hydroxycobalamin. The oil-soluble nutritional supplement typically includes at least one oil-soluble vitamin. Some examples of oil-soluble vitamins include vitamin A, vitamin E, vitamin D, and vitamin K, and active derivatives thereof.

In a first step (i.e., step (i)) of the process, a dispersion of spheroidal polysaccharide-stabilized complexes (SPSCs) containing a water-soluble polysaccharide (first polysaccharide) and at least one water-soluble nutritional supplement (WSNS) in an aqueous medium is produced. In the SPSCs, the water-soluble polysaccharide and WSNS are complexed with each other by ionic interactions. The term “spheroidal,” as used herein, refers to a shape that is precisely or approximately spherical in shape. An approximately spherical shape may be, for example, an oval or globular shape. The water-soluble polysaccharide (i.e., “first polysaccharide”) in step (i) may be any edible polysaccharide that is substantially or completely soluble in water or an aqueous solution, such as, for example, an aminated polysaccharide (e.g., chitosan), carboxymethylcellulose, dextran, pectin, guar gum, xanthan gum, locust bean gum, gum arabic, or carrageenan, or a combination thereof. In particular embodiments, the water-soluble polysaccharide in step (i) is chitosan or other aminated polysaccharide, e.g., one containing glucosamine units, or a derivative thereof. Preferably, the chitosan or other aminated polysaccharide is plant-based and allergen-free. Preferably, the chitosan or other aminated polysaccharide is not derived from shrimp or other crustacean. The WSNS may be selected from for example, one or more water-soluble vitamins (e.g., vitamin C and/or one or more B vitamins), provitamins, and/or one or more water-soluble minerals, amino acids, proteins, phytochemicals, anti-oxidants, anthocyanins, enzymes, and/or co-enzymes.

The process for producing the SPSCs in step (i) can be any process known in the art, or subsequently developed, capable of sufficient integral (i.e., intimate) mixing and integration of the at least one WSNS and the water-soluble polysaccharide in solution so as to produce a complex (i.e., SPSC) in which the water-soluble polysaccharide and WSNS are complexed with each other by ionic interactions. In some embodiments, the process is a single-step or one-pot preparation in which the at least one WSNS and the water-soluble polysaccharide are together combined in water or an aqueous solution before being integrally mixed. In other embodiments, step (i) is a three-step process comprising: (i-1) preparing a first aqueous solution containing at least one WSNS, (i-2) preparing a second aqueous solution containing the water-soluble polysaccharide, and (i-3) mixing the first and second aqueous solutions to form the SPSCs. The term “integral mixing” includes any of the methods known in the art of mixing two or more solid components into a liquid to form a solution or emulsion of the components. The method may be, for example, mechanical or hand stirring, vortex mixing, agitation or tumbling mixing, sonication, or ultrasonication, or any combination thereof, such as a combination of any two of these. The SPSCs have a microscopic size, such as a size of 2 nm, 3 nm, 4 nm, 5 nm, 10 nm, 20 nm, 30 nm, 40 nm, 50 nm, 100 nm, 150 nm, 200 nm, 250 nm, 500 nm, or a size within a range bounded by any two of the foregoing values (e.g., 2-100 nm, 2-50 nm, 2-10, 2-5, 2-3, 5-100 nm, 5-50, or 5-10 nm, etc.) or bounded by any intermediary value(s) therebetween (e.g., 6-9 nm, 3-49 nm, etc.). The at least one WSNS is typically present in an amount of 1 mg/mL to 100 mg/mL by volume of the aqueous medium. In different embodiments, the at least one WSNS is present in an amount of precisely or about 1, 2, 5, 10, 20, 30, 40, 50, 60, 70, 80, 90, or 100 mg/mL by volume of the aqueous medium, or alternatively in an amount in a range bounded by any two of the foregoing values or bounded by any intermediary value(s) therebetween.

Generally, one or both of the first and second aqueous solutions in step (i) are adjusted to a pH of precisely or about 2, 3, 4, 5, 6, 7, or 8, or a pH within a range bounded by any two of these values (e.g., 2-8, 2-7, 2-6, 3-8, 3-7, 3-6, 4-8, 4-7, 4-6, 2.1-7.9, etc.). The resulting solution containing the SPSCs in step (i) preferably has any of the foregoing pH values or ranges thereof. In particular embodiments, the pH of the first and/or second aqueous solutions or the resulting solution containing the SPSCs has a pH of precisely or about 5 (e.g., 4.5-5.5 or 4.5-5). Moreover, the first and/or second aqueous solutions may contain any one or more non-toxic pH adjusting chemicals, such as an acid (e.g., HCl) or base (NaOH) or a salt formed by reaction between these. The resulting solution containing the SPSCs in step (i) may or may not further contain one or more non-toxic buffers, surfactants, or emulsifying agents (e.g., lecithin).

In a second step (i.e., step (2)) of the process, an oil-supplement solution containing at least one oil-soluble nutritional supplement (OSNS) dissolved in an edible (food grade) oil is prepared. The OSNS may be, for example, one or more oil-soluble vitamins (vitamin A, vitamin E, vitamin D, and/or vitamin K), pro-vitamins, amino acids, proteins, phytochemicals, anti-oxidants, anthocyanins, fatty acids, and/or oil extracts. The food grade oil can be any of the food grade oils known in the art. The food grade oil may be for human or animal consumption. The food grade oil may contain, for example, a mono-, di-, or tri-glyceride, or combination thereof, and may be, for example, a plant or animal derived oil. Some examples of plant (vegetable) oils include coconut oil, corn oil, canola oil, palm oil, grape seed oil, soybean, peanut oil, olive oil (e.g., extra virgin olive oil), almond oil, avocado oil, cottonseed oil, flax seed oil, sesame seed oil, walnut oil, safflower oil, sunflower oil, palm kernel oil, hemp seed oil, grape seed oil, rapeseed oil, lemon oil, cocoa butter, and orange oil, any of which may be refined or unrefined. Alternatively, the food grade oil may be or include an artificial food grade oil, such as mineral oil or a fatty acid-substituted sugar (e.g., olestra). Alternatively, the food grade oil may be or include a fish, krill, or algal oil, which are typically high in omega-3 fatty acids. In some embodiments, any one or more of the foregoing types of oils may be excluded from the composition. In some embodiments, the oil has a low moisture content, preferably no more than or less than 0.3, 0.2, 0.1, 0.05, 0.02, or 0.01% of water. The at least one OSNS is typically present in an amount of 1 mg/mL to 100 mg/mL by volume of the oil. In different embodiments, the at least one OSNS is present in an amount of precisely or about 1, 2, 5, 10, 20, 30, 40, 50, 60, 70, 80, 90, or 100 mg/mL by volume of the oil, or alternatively in an amount in a range bounded by any two of the foregoing values or bounded by any intermediary value(s) therebetween.

In a third step (i.e., step (3)) of the process, the dispersion of SPSCs, as produced in step (i), is integrally mixed (e.g., blended) with the oil-supplement solution, as produced in step (ii), to produce the nutritional multi-supplement complex described above containing at least one water-soluble nutritional supplement and at least one oil-soluble nutritional supplement. The term “integral mixing” includes any of the methods known in the art of mixing two or more solid components into a liquid to form a solution or emulsion of the components. The method may be, for example, mechanical or hand stirring, vortex mixing, agitation or tumbling mixing, sonication, or ultrasonication, or any combination thereof, such as a combination of any two of these. By virtue of the mixing/blending process, the nutritional multi-supplement complex contains droplets of the oil-supplement solution encapsulated by the SPSCs. The droplets of the oil-supplement solution have a microscopic size, such as a size of 2 nm, 5 nm, 10 nm, 20 nm, 30 nm, 40 nm, 50 nm, 100 nm, 150 nm, 200 nm, 250 nm, 500 nm, 1 micron, 2 microns, or 5 microns, or a size within a range bounded by any two of the foregoing values (e.g., 2-1000 nm, 2-500 nm, 2-100 nm, 2-50 nm, 5-1000 nm, 5-500 nm, 5-100 nm, 5-50 nm, 10-1000 nm, 10-500 nm, 10-100 nm, 10-50 nm, 20-1000 nm, 20-500 nm, 20-100 nm, 20-50 nm, 50-1000 nm, 50-500 nm, 50-100 nm, 100-1000 nm, 200-1000 nm, or 500-1000 nm) or bounded by any intermediary value(s) therebetween. In some embodiments, the size of the SPSCs are smaller than the size of the droplets of the oil-supplement solution. To yield the encapsulated arrangement, the mixing/blending process should be of sufficient power to disperse and intermix the SPSCs with the OSNS on a microscopic level. In preferred embodiments, the mixing/blending process includes ultrasonication. The ultrasonication may employ a power of precisely, about, or at least, for example, 500 W, 600 W, 700 W, 800 W, 900 W, or 1000 W, or a power in a range between about 500 W-1000 W, and a frequency of precisely, about, or at least, for example, 5 kHz, 10 kHz, 20 kHz, 30 kHz, 40 kHz, or 50 kHz, or a frequency in a range between about 5 kHz-50 kHz.

In some embodiments, the process includes an additional step (e.g., step (iv)) of mixing the nutritional multi-supplement complex with a second polysaccharide to form a polysaccharide-encapsulated multi-supplement complex. The second polysaccharide may be the same or different as the polysaccharide (i.e., “first polysaccharide”) used in step (i) to make the SPSCs. The second polysaccharide may be selected from any of the polysaccharides recited earlier above for the first polysaccharide. The second polysaccharide preferably functions to form a durable yet dissolvable coating around the multi-supplement complexes with acceptable physical properties making it amenable to subsequent spray-drying. In particular embodiments, the second polysaccharide is or includes a dextrin, such as maltodextrin. In other embodiments, the second polysaccharide may alternatively be or include a gum, such as xanthan gum. In other embodiments, a gum, such as xanthan gum, may be excluded as the second polysaccharide. In some embodiments, the polysaccharide-encapsulated multi-supplement complex is spray-dried to form solid particles of the polysaccharide-encapsulated multi-supplement complex. The resulting solid particles may have a size of, for example, precisely or at least 200 nm, 250 nm, 300 nm, 400 nm, 500 nm, 600 nm, 1 micron, 2 microns, 5 microns, 10 microns, 15 microns, 20 microns, 50 microns, 100 microns, 150 microns, 200 microns, 250 microns, 300 microns, 350 microns, 400 microns, or 500 microns, or a size within a range bounded by any two of the foregoing values (e.g., 200-1000 nm, 200-600 nm, 300-1000 nm, or 300-600 nm) or bounded by any intermediary value(s) therebetween.

The resulting nutritional multi-supplement complex, as prepared by the above method, contains droplets of an oil-supplement solution encapsulated by SPSCs, wherein: (a) the oil-supplement solution comprises at least one oil-soluble nutritional supplement (OSNS) dissolved in a food grade oil, and (b) the SPSCs contain a water-soluble polysaccharide and at least one water-soluble nutritional supplement (WSNS) in an aqueous medium, wherein the water-soluble polysaccharide and WSNS are complexed with each other by ionic interactions in the SPSCs. In some embodiments, as described above, the nutritional multi-supplement complex is encapsulated by a second polysaccharide, such as a dextrin, or more particularly, maltodextrin, or a gum. The encapsulated multi-supplement complex is typically spray dried to convert it to a solid (spray-dried) particulate form having any of the particle sizes described above.

In another aspect, the present disclosure is directed to a beverage containing the nutritional multi-supplement complex described above, wherein the nutritional multi-supplement complex may or may not be encapsulated. In the beverage, the nutritional multi-supplement complex is homogeneously dispersed in an aqueous medium suitable for human consumption. The beverage may have any natural or artificial flavoring, including any one or more natural or artificial sweeteners. The beverage may be, for example, a tea beverage, which may contain one or more components from a tea plant. The beverage may be carbonated or non-carbonated. The beverage may also contain alcohol or be non-alcoholic. The beverage may also be caffeinated or non-caffeinated. The beverage may also contain dairy or be dairy-free. The beverage may also be a concentrate designed to be diluted by the user. The beverage may also contain one or more additional components, such as one or more preservatives, pH modifying agents, emulsion stabilizers, colorants, or salts. In other embodiments, any one of the foregoing additional components may be excluded from the beverage.

In another aspect, the present disclosure is directed to a tea bag in which the nutritional multi-supplement complex, as described above, is included. Typically, the nutritional multi-supplement complex in the tea bag is coated (encapsulated) with a second polysaccharide, such as maltodextrin and/or a gum, as described earlier above. In some embodiments, the nutritional multi-supplement complex is contained within a tea bag enclosure, either alone or in combination with one or more other components. The one or more other components may include, for example, material from a botanical source (typically dried plant matter), such as a leaf, flower, or root of a plant useful in making a tea. In some embodiments, the botanical source includes one or more components from a tea plant, such as leaf, flower, or root of a tea plant. In some embodiments, the botanical source is an herb (e.g., chamomile, lavender, ginger, or ginseng). In some embodiments, the botanical source is a plant extract. In some embodiments, the tea bag enclosure has a pore size cut-off of precisely or about 10 microns, 15 microns, 20 microns, 50 microns, 100 microns, 150 microns, 200 microns, 250 microns, 300 microns, 350 microns, 400 microns, or 500 microns, or a pore size cut-off within a range bounded by any two of the foregoing values or bounded by any intermediary value(s) therebetween. The term “about,” as used herein is generally within 10%-20% of a given value.

Preferably, the pore size cut-off of the tea bag is lower than the size of the spray-dried particles to ensure that the particles do not fall through pores of the tea bag enclosure prior to steeping. The steeping may be hot or cold steeping. Moreover, after steeping, the resulting solution (e.g., water, flavored drink, or tea) is preferably substantially or completely devoid of turbidity or opacity, or conversely, substantially or completely translucent. The spray-dried particle sizes and pore size cut-off of the tea bag can be selected to ensure low or no turbidity in the resulting solution after steeping.

In another aspect, the present disclosure is directed to a solid food product containing any of the nutritional multi-supplement complexes described above, wherein the nutritional multi-supplement complex may be produced by any of the methods described above. The term “containing,” as used herein, includes direct incorporation (e.g., injection or impregnation), coating, or mixing (e.g., blending) of the nutritional multi-supplement complex into or onto the solid food product or intermediary product used to make the solid food product. The food product may be, for example, a fruit, fruit-containing product, fruit-derived product, grain, grain-containing product, grain-derived product, vegetable, vegetable-containing product, vegetable-derived product, meat, meat-containing product, meat-derived product, baked good (e.g., bread, cake, or cookie), or refrigerated good (e.g., pudding, sour cream, kefir, smoothie, or ice cream).

In another aspect, the present disclosure is directed to a capsule or tablet form of the nutritional multi-supplement complex described above. The capsule or tablet form includes at least the nutritional multi-supplement complex described above. Typically, the nutritional multi-supplement complex in the capsule or tablet is coated (encapsulated) with a second polysaccharide, such as maltodextrin or a gum, as described earlier above.

Examples have been set forth below for the purpose of illustration and to describe the best mode of the invention at the present time. However, the scope of this invention is not to be in any way limited by the examples set forth herein.

### EXAMPLES

### Example 1

Preparation of Multi-Vitamin Carriers (Chitosan-Stabilized Oil-In-Water (O/W) Emulsions)

Chitosan-vitamin complexes were first prepared as follows: 500 mg of anthocyanin, 500 mg of VC, 500 mg of VB6 and 42.5 mg of VB12 were dissolved together in 50 mL deionized water of pH 5. Chitosan was separately dissolved in acetic acid solution (1%, v/v) under magnetic stirring, followed by filtration (filter pore size 50 um) to remove insoluble compounds. The pH of the chitosan solution was adjusted to 5.0 using hydrochloric acid or sodium hydroxide. The chitosan-vitamin complexes were then formed by mixing 50 mL of chitosan solution and 50 mL of vitamin solution at pH 5 under magnetic stirring for 30 min at room temperature. To prepare the chitosan-stabilized emulsions, canola oil containing 1 mL VE and 47.1 mg VA was added into the obtained chitosan-vitamin complexes. The mixture was then subjected to ultrasonication using a 750 W ultrasonic processor with a high-power sonic tip operated at 20 kHz frequency. The system was sonicated for 10 minutes in an ice bath at an acoustic intensity of 300 W cm⁻² (5 s on, 2 s off). The resulting chitosan-stabilized oil-in-water (O/W) emulsions can function as multi-vitamin carriers. Finally, 8 g maltodextrin was added into the carriers. The addition of maltodextrin can assist in the formation of multiple layers around the multi-vitamin carriers when spray dried, increase water solubility when re-dispersed, and favor the flowy powder formation.

Production of Spray-Dried Multi-Vitamin Carriers

The drying of the emulsion samples was carried out using a spray dryer equipped with an atomizer nozzle using a product feed temperature of 4° C.; inlet and outlet temperature of 160° C. and 60-70° C., respectively. The dried powders were removed from the dryer and stored until they were evaluated. The end product is referred to as multi-vitamin powder. The final loadings were 4.9%, 4.9%, 0.42%, 9.9%, and 0.46% for VC, VB6, VB12, VE, and VA, respectively.

Visual Observation

All spray-dried samples were observed visually and recorded as photography by digital camera.

Release of Vitamins During Tea Bag Steeping Process

To evaluate the release of encapsulated vitamins from the spray-dried powder into water, 30 mg spray-dried powders was placed in the tea bag, which was then immersed in a beaker containing 30 mL of deionized water (pH 5). The beaker was placed in a shaker and shaken slowly in a water bath at 4° C. or 100° C. At appropriate brewing time intervals, an aliquot of solution (0.5 mL) was removed with a pipette for measuring the released vitamins. To determine the amount of water-soluble vitamins, the pH of solution was adjusted to pH 1, followed by filtration (filter pore size 50 μm). The filtrate was collected and tested for the released water-soluble vitamins using a UV-2600 spectrophotometer (at 245 nm for VC, 291 nm for VB6, and 361 nm for VB12, respectively. To determine the amount of oil-soluble vitamins, the pH of solution was adjusted to pH 1 and vortexed. Then, 3 mL organic solvent (chloroform+methanol, 1:1, v/v) was added into the solution to extract the oil-soluble vitamins. After centrifugation at 1000 g for 5 min, the layer of chloroform was removed and tested for the released oil-soluble vitamins using the spectrophotometer at 295 nm for VE and 326 nm for VA, respectively. The release rate was determined by the following equation: release rate (%)=(released vitamins/initially added vitamins)×100.

Results and Discussion

Strategy for the Fabrication of Multi-Vitamin Carriers

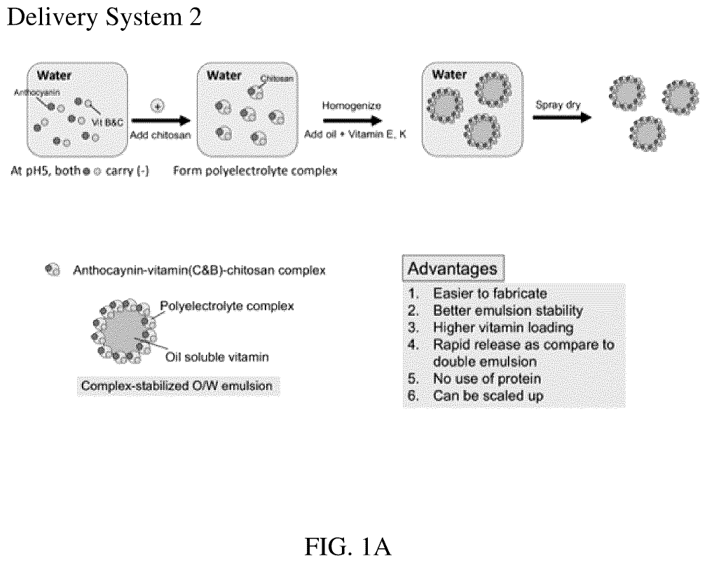

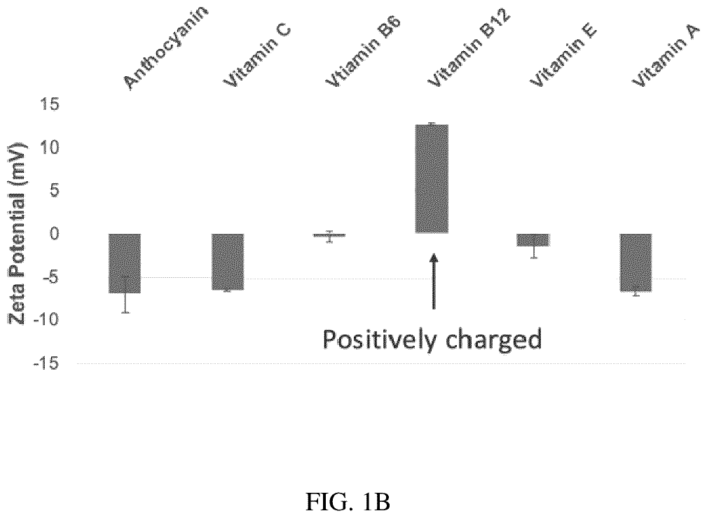

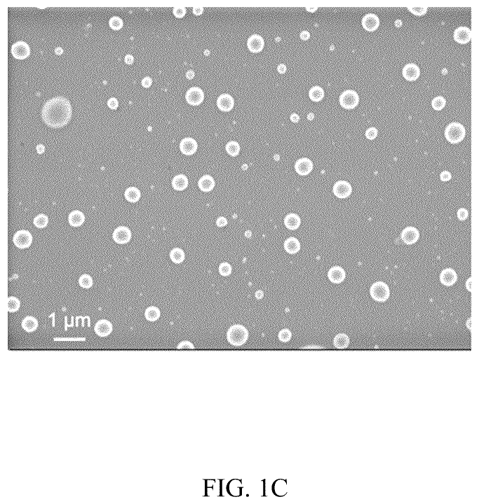

A facile, green, and powerful strategy was explored to engineer a robust O/W emulsion for co-delivery of water-soluble vitamins (VC, VB6, VB12) and oil-soluble vitamins (VA, VE). The preparation involved the complexation of chitosan and water-soluble vitamins and subsequent formation of chitosan-stabilized O/W emulsions. The approach is illustrated in FIG. 1A. First, the chitosan-vitamin complexes were prepared by mixing solutions of chitosan, anthocyanins, and water-soluble vitamins in deionized water at pH 5. At this pH, chitosan is positively charged, while anthocyanins and vitamins are negatively charged, except for vitamin B12 (FIG. 1A). Thus, as shown in FIG. 1B, they can form complexes via electrostatic interaction once they are mixed together. The resulting chitosan-vitamin complexes were mixed with canola oil containing oil-soluble vitamins. After homogenization by ultrasonication, the chitosan-stabilized emulsions can form as optically opaque homogenous suspensions. In the ultrasonication process, the chitosan-vitamin complexes tended to coat on the surface of oil droplets, providing a physical layer for stabilization of oil-soluble vitamins.

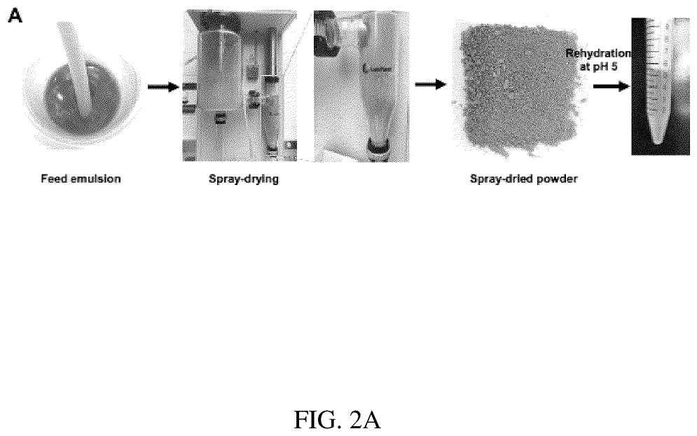

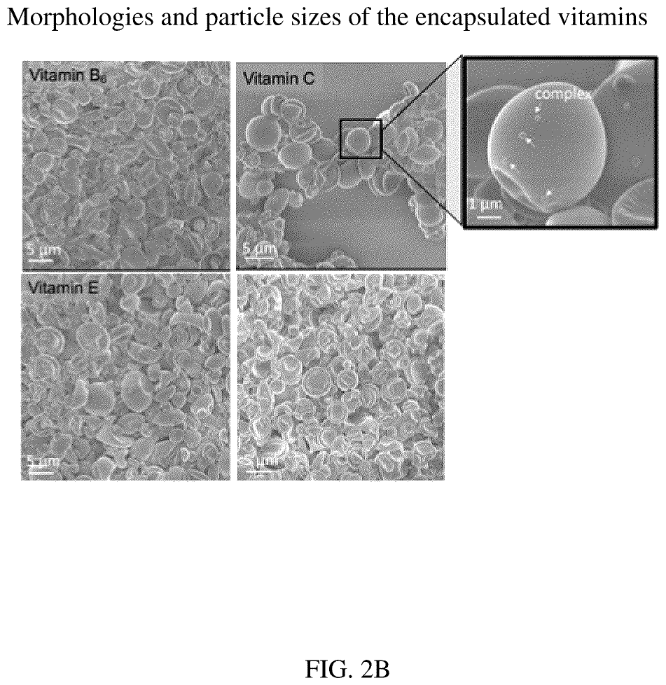

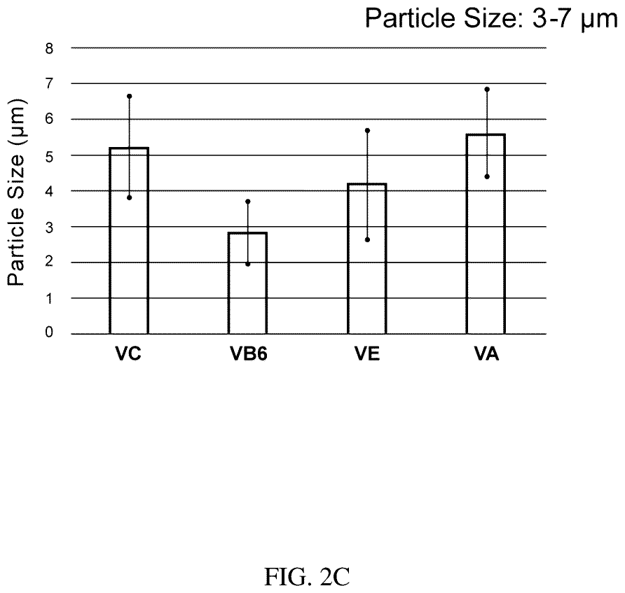

After dissolving maltodextrin in the chitosan-stabilized emulsions, the emulsions can be spray-dried at appropriate parameters to result in flowy and reddish powders (FIG. 2A). The morphologies of the powdered encapsulated vitamins were then examined under the scanning electron microscope, showing smaller complexes on the encapsulated vitamin powders' surface (FIG. 2B, for each vitamin). These complexes are believed to be the polyelectrolyte complex formed between chitosan, water-soluble vitamins, and anthocyanins. The powder was rapidly dissolved in deionized water of pH 5 with almost transparent appearance, and exhibited a size of roughly 3-7 μm (FIG. 2C). This indicated the high potential for fast release of vitamins from a tea bag into the external aqueous solution, as the size of the tea bag cut is around 200 μm.

Without being bounded by theory, it is believed that the cohesion and strength of the complexes is in large part a result of electrostatic attraction between positively charged ions of chitosan and negatively charged vitamins B6 and A and C. The value of the zeta potential (mV) indicates the degree of electrostatic repulsion and is the indicator/predictor of the stability of the dispersed particles. The absolute value difference of positive and negative electrostatic charges, measured in mV, between vitamins and chitosan is not the only factor that keeps the vitamins on the interface. For example, the spray-drying process helps to stabilize V B12 on the surface of the droplets even though it is slightly positively charged.

Release of Water-Soluble Vitamins During Tea Bag Steeping Process (pH 5, 4° C.)

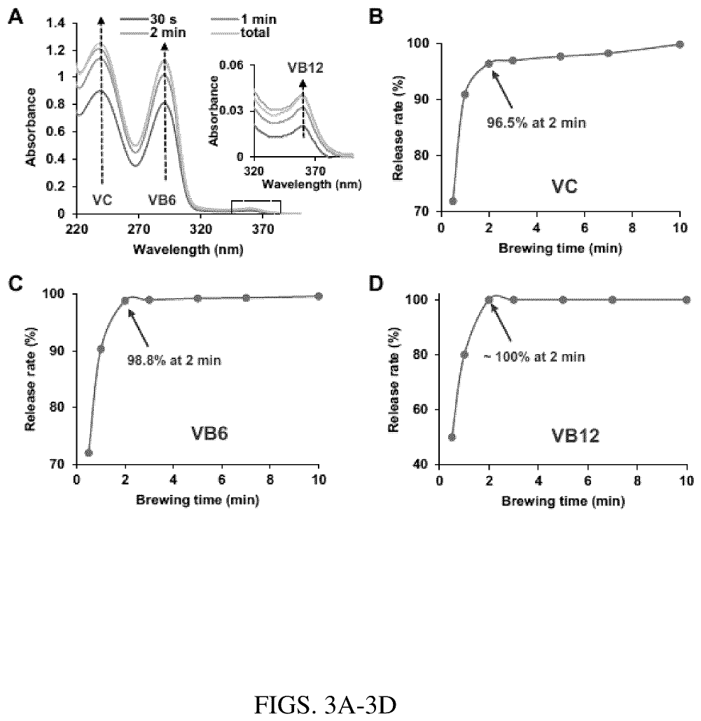

The spray-dried powders were placed into the tea bag, which was then immersed in the deionized water of pH 5 at 4° C. The release of vitamins was monitored by UV absorption spectra of the external water. FIG. 3A shows the spectral changes of UV absorption of water-soluble vitamins (VC, VB6, and VB12) as a function of brewing time.

FIG. 3A clearly shows the maximum absorption peaks for all vitamins. With increasing brewing time, the absorbance of vitamins increased, indicating the progressive release of vitamins from tea bag into the outer solution. FIGS. 3B-D show that all VC, VB6, and VB12 exhibited a burst release, with 96.5%, 98.8%, and 100% of the total amounts released during the initial 2-minute brewing period. These results demonstrated that the water-soluble vitamins (B6, B12 and C) released well in 10-min brewing period at 4° C. (pH 5), releasing up to 96%-99.9%. At 100° C., the release should occur even faster.

Release of Oil-Soluble Vitamins During Tea Bag Steeping Process (pH 5, 4° C. and 100° C.)

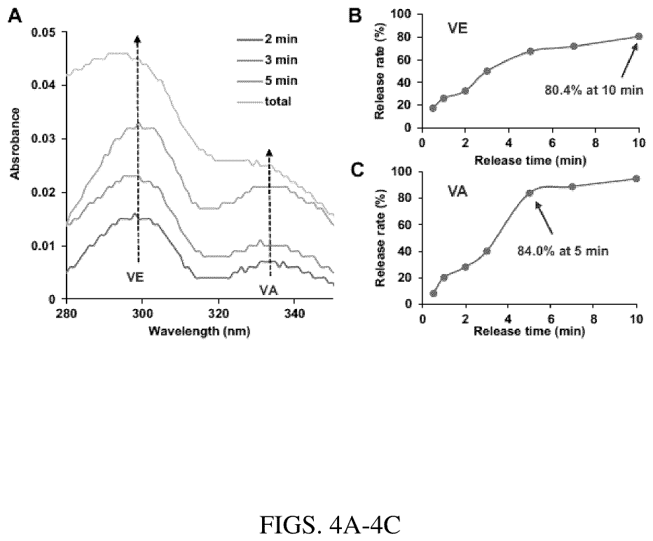

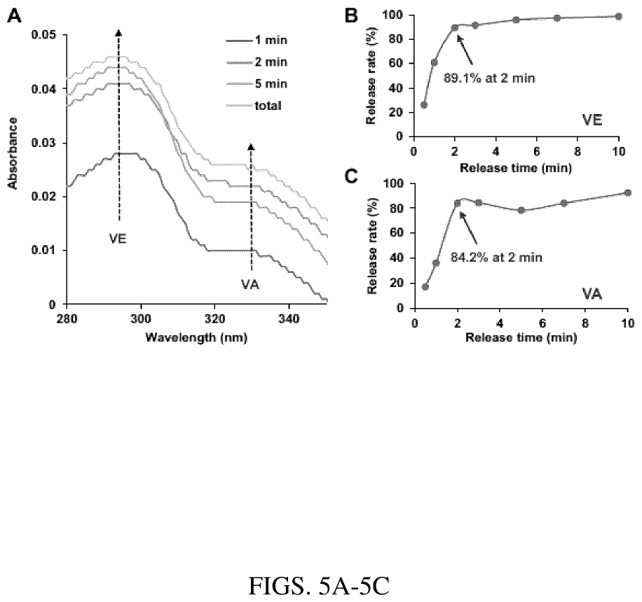

For the release of oil-soluble vitamins, their UV spectral changes were also monitored. FIG. 4A shows that the absorption peaks of VE and VA can be detected at the maximum wavelengths of around 295 and 326 nm, respectively. At 4° C., the oil-soluble vitamins released slower than the water-soluble ones (FIGS. 4B-4C). This is because the hydrophobicity nature of oil-soluble vitamins slowed their diffusion in the hydrophilic environment. As a result, they followed a sustained release pattern with the release rates of 80.4% at 10 min for VE and 84.0% at 5 min for VA. At 100° C., the release was accelerated for all oil-soluble vitamins (FIG. 5). Roughly 90% encapsulated vitamin E and A are being released within 4 min. Based on these results, it can be concluded that the multi-vitamin carriers feature excellent stability and water solubility, which permits quick release of both water-soluble and oil-soluble vitamins within a 10-min brewing period, regardless of the brewing temperature.

### Example 2

Exploration of Another Approach for Multivitamin Encapsulation: Creation of a Water-In-Oil Emulgel Carrier

Vitamin solutions were first prepared as follows: 25 mg of anthocyanin, 25 mg of VC, 25 mg of VB6 and 25 mg of VB12 were dissolved together in 50 mL deionized water. This vitamin solution was emulsified in corn oil containing 1% of glycerol monooleate and 20 mg of VA and VE at a ratio of 2:8 (v/v). Subsequently, this primary emulsion was further emulsified in a 1% locust bean gum solutions at 2:8 (v/v) ratio. To spray-dry this solution, 4 w % of maltodextrin was added and the emulsion subjected to a spray dryer equipped with an atomizer nozzle with a product feed temperature of 4° C. and inlet and outlet temperatures of 160° C. and 60-70° C., respectively. The dried powders were removed from the dryer and stored until they were evaluated. The end product is referred to as the multi-vitamin powder.

Results

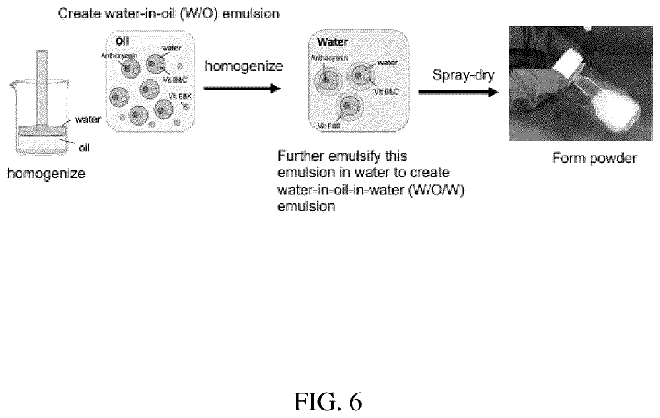

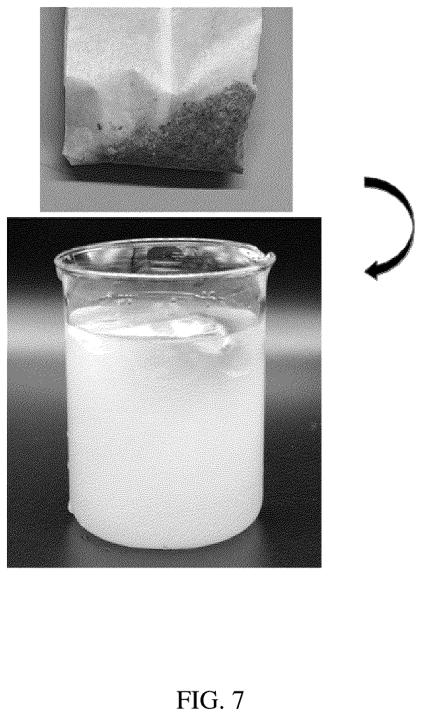

In this part, a water-in-oil-in-water (W/O/W) emulsion was explored for co-delivery of water-soluble vitamins (VC, VB6, VB12) and oil-soluble vitamins (VA, VE). The preparation involves first the suspension of water-soluble vitamin in oil containing oil-soluble vitamin, followed by a subsequent suspension of such W/O emulsion in another body of oil. The resulting W/O/W emulsion was then subjected to a spray-drying process with addition of maltodextrin for powder production. The approach is illustrated in FIG. 6. Although this encapsulated multi-vitamin powder seemed to suspend readily in the tea during brewing process, the brewed tea resulted in a highly turbid beverage, as shown in FIG. 7. Therefore, this encapsulated multi-vitamin powder may be appropriate for uses where high turbidity is acceptable, or where high turbidity may be desired.

### Example 3

Method for Quantitative Determination of the Water-Soluble Vitamins (Vitamin C, Vitamin B6, Vitamin B12) from the Encapsulated Carrier During the Tea Bag Steeping Process

Reagents:
HCl, for pH adjustments
NaOH, for pH adjustments
Water, in house deionized water
Vitamin C, from DSM, used as compound standard
Vitamin B6, from DSM, used as compound standard
Vitamin B12, obtained commercially, used as compound standard

Standard Solutions of Vitamins

Vitamin C, vitamin B6, vitamin B12 solutions were prepared in deionized water at a pH of 1 at different concentrations to establish standard curves (calibration curve) using a UV-spectrophotometer. The standard curves should establish a linear correlation. Known concentrations of the vitamin samples should be plotted as the x-axis, and the corresponding measurement of the absorbance on the y-axis, which establishes an equation of y=mx+b of the corresponding compound (wherein m is the slope and b the y-intercept).

Sample Preparations

The samples to be tested (encapsulated vitamin carriers, 30 mg) were loaded into the tea bags along with tea leaves, followed by steeping in 30 mL of deionized water (4° C. or 100° C.) at pH 5. At appropriate steeping time intervals, an aliquot of solution (0.5 mL) was removed with a pipette for measuring the released vitamins. To determine the amount of water-soluble vitamins, the pH of solution was adjusted to pH 1, followed by filtration (filter pore size 50 μm). The filtrate was then collected and quantified using a UV-spectrophotometer for absorbance. A blank was prepared using deionized water at pH of 1 (adjusted by HCl).

Measurement Conditions

Using a UV-spectrophotometer, the absorbance of Vitamin C was measured at a wavelength of 245 nm. The absorbance of Vitamin B6 was measured at a wavelength of 291 nm. The absorbance of Vitamin B12 was measured at a wavelength of 361 nm.

Calculations

According to the corresponding standard equation, the measured vitamin absorbance (A) is plotted to find the vitamin concentration.
Vitamin C released: Avc-Bvc/Svc
Vitamin B6 released: AvB6-BvB6/SvB6
Vitamin B12 released: AvB12-BvB12/SvB12

Release rate (%)=(released vitamins/initially vitamins in the tea bags)×100
Where:
Avc=Absorbance response of vitamin C in the sample preparation
AvB6=Absorbance response of vitamin B6 in the sample preparation
AvB12=Absorbance response of vitamin B12 in the sample preparation
Bvc=Y-intercept of vitamin C standard curve
BvB6=Y-intercept of vitamin B6 standard curve
BvB12=Y-intercept of vitamin B12 standard curve
Svc=Slope of vitamin C standard curve
SvB6=Slope of vitamin B6 standard curve
SvB12=Slope of vitamin B12 standard curve

### Example 4

Method for Quantitative Determination of the Oil-Soluble Vitamins (Vitamin a, Vitamin E) from the Encapsulated Carrier During the Tea Bag Steeping Process

Reagents
HCl, for pH adjustments
NaOH, for pH adjustments
Water, in house deionized water
Chloroform
Methanol
Vitamin A (retinol palmitate), from Sigma-Aldrich, used as compound standard
Vitamin E (tocopherols, mixed, FG), from Sigma-Aldrich, used as compound standard

Standard Solutions of Vitamins

Vitamin A and Vitamin E solutions were prepared in organic solvent (chloroform+methanol, 1:1, v/v) with different vitamin concentrations. After centrifugation at 1000 g for 5 min, the layer of chloroform was removed and measured to establish standard curves (calibration curve) using UV-spectrophotometer. The standard curves should establish a linear correlation. Known concentration of the vitamin samples should be plotted as the x-axis, and the corresponding measurement of the absorbance on the y-axis, which establishes an equation of y=mx+b of the corresponding compound (wherein m is the slope and b the y-intercept).

Sample Preparations

The samples to be tested (encapsulated vitamin carriers, 30 mg) were loaded into the tea bags along with tea leaves, followed by steeping in 30 mL of deionized water (4° C. or 100° C.) at pH 5. At appropriate steeping time intervals, an aliquot of solution (0.5 mL) was removed with a pipette for measuring the released vitamins. To determine the amount of oil-soluble vitamins, the pH of the solution was adjusted to a pH of 1, followed by filtration (filter pore size 50 μm). Then, 3 mL organic solvent (chloroform+methanol, 1:1, v/v) was added into the solution to extract the oil-soluble vitamins. After centrifugation at 1000 g for 5 min, the layer of chloroform was removed and quantified using the UV-spectrophotometer for absorbance. A blank was prepared using chloroform.

Measurement Conditions

Using a UV-spectrophotometer, the absorbance of Vitamin A was measured at a wavelength of 326 nm. The absorbance of Vitamin E was measured at a wavelength of 295 nm.

Calculations

According to the corresponding standard equation, measured vitamin absorbance (A) was plotted to find the vitamin concentration.
Vitamin E released: AvE-BvE/SvE
Vitamin A released: AvA-BvA/SvA

Release rate (%)=(released vitamins/initially vitamins in the tea bags)×100
Where:
AvE=Absorbance response of vitamin E in the sample preparation
AvA=Absorbance response of vitamin A in the sample preparation
BvE=Y-intercept of vitamin E standard curve
BvA=Y-intercept of vitamin A standard curve
SvE=Slope of vitamin E standard curve
SvA=Slope of vitamin A standard curve

In summary, the above experiments have demonstrated a multi-vitamin carrier with improved water dispersibility, including both water-soluble vitamins (B6, B12 and C) and oil-soluble vitamins (E&A). Water soluble vitamins (B6, B12 and C) release well in a 10-minute brewing period at 4° C. (pH=5), releasing up to 96%-99.9%. At 100° C., the release should occur even faster. Oil-soluble vitamins (E & A) release slower than the water-soluble ones. At 4° C., oil soluble vitamins can release up to 80-90% in 10 minutes. At 100° C., roughly 90% encapsulated vitamin E and A are being released within 4 minutes.

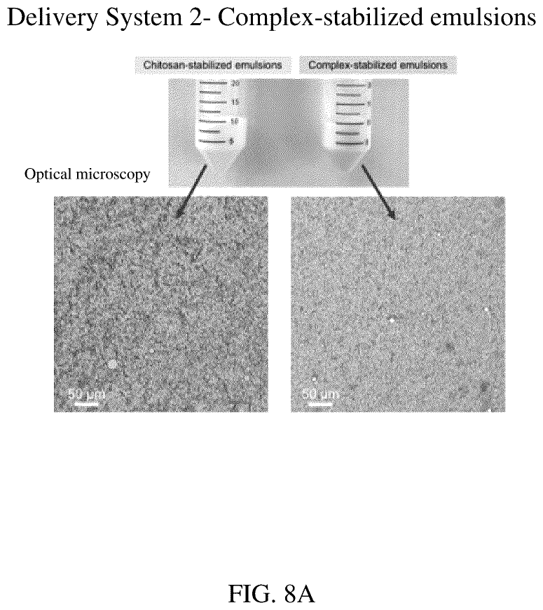

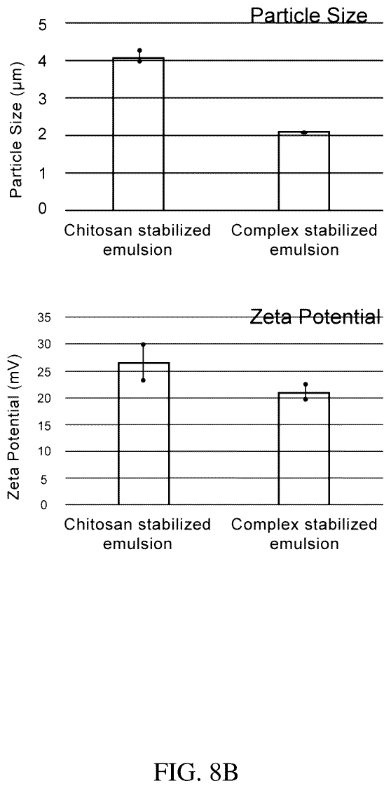

FIG. 8A shows optical microscopy of System 2 complex-stabilized emulsions. The left field shows emulsion particle size stabilized by chitosan, while the right field shows emulsion stabilized by polyelectrolyte complex. FIG. 8B (top) is a bar graph showing particle size reduction when comparing chitosan-stabilized and complex-stabilized emulsions in the more advanced processing phase. FIG. 8B (bottom) is bar graph showing zeta potential change (mV) between chitosan-stabilized and complex-stabilized emulsions.

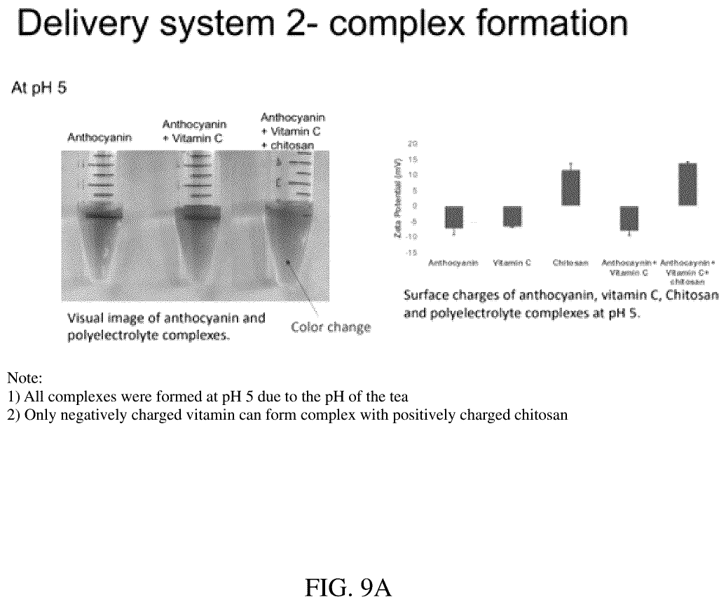

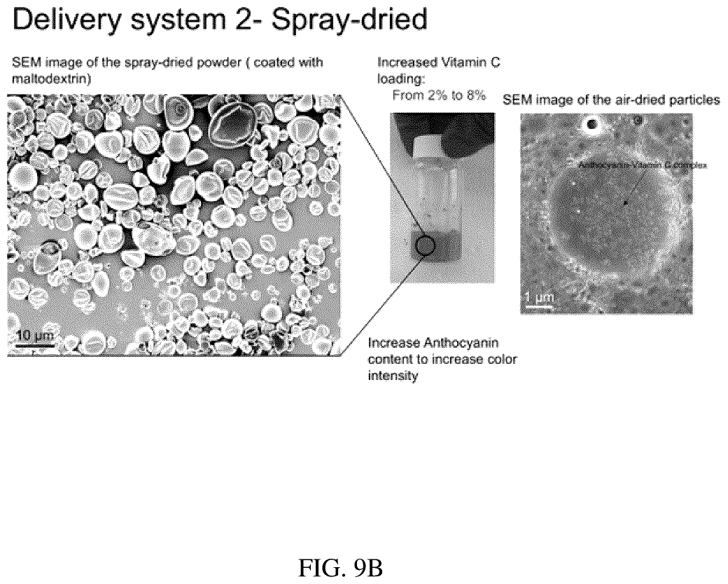

FIG. 9A (left) is a visual image showing gradual color change of the complex after adding anthocyanin, vitamin C, and chitosan at pH=5. FIG. 9A (right) is a bar graph showing changing surface charges forming polyelectrolyte complexes at pH=5. FIG. 9B (left) is a SEM image of the spray-dried powder, with maltodextrin coating. FIG. 9B (right) is a SEM image of the air-dried particles of anthocyanin plus vitamin C complex.

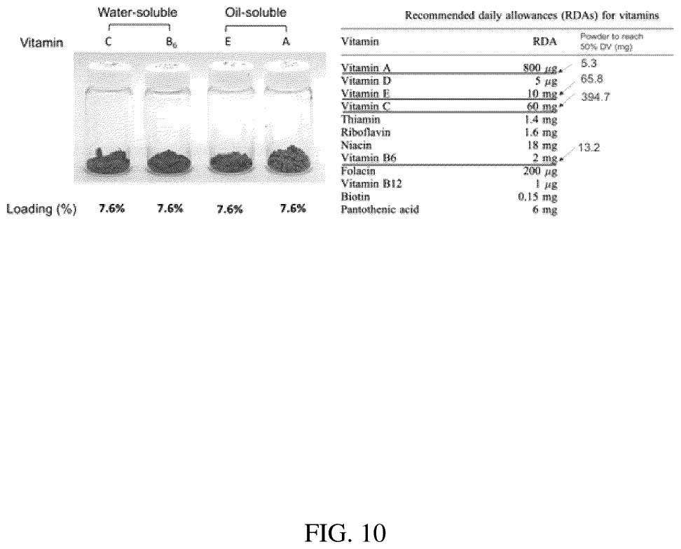

In FIG. 10, the photo on left shows snapshots of perfecting encapsulation efficiency during spray drying, wherein the samples include spray dried particles containing maltodextrin, with water soluble vitamins (C and B6) and oil soluble vitamins (E and A) obtained with 7.6% loading by weight. The table on right shows calculations between milligrams of powder and the individual vitamins, each of them to target 50% RDA; testing encapsulation efficiency in progress.

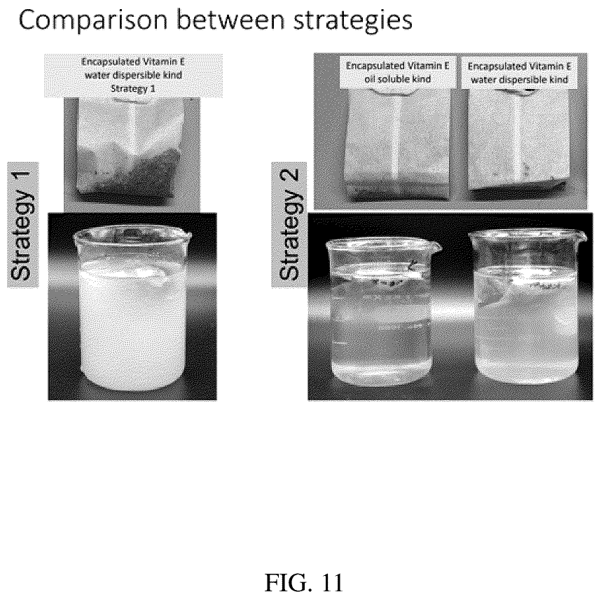

FIG. 11 shows photographs comparing Strategy 1 and Strategy 2. Left photos show results of Strategy 1, fabricating double layer W/O/W emulsion with high density internal phase: the encapsulated water dispersible (pre-treated) oil soluble vitamin E resulting turbidity in the final beverage. Right photos show results of Strategy 2: emulsion fabrication with the cross-linking, preferred method: Left side of Strategy 2: encapsulating oil-soluble vitamin E (mixed tocopherols, food grade) resulting a clear green tea beverage. Right side of Strategy 2: encapsulated water-dispersible (pre-treated) oil-soluble vitamin E with green tea in the blend, resulting in turbidity in the ready-to-drink beverage.

While there have been shown and described what are at present considered the preferred embodiments of the invention, those skilled in the art may make various changes and modifications which remain within the scope of the invention defined by the appended claims and the examples below.

What is claimed is:

1. A method for preparing a nutritional multi-supplement complex, the method comprising:
(i) preparing a dispersion of spheroidal polysaccharide-stabilized complexes comprising a water-soluble polysaccharide and at least one water-soluble nutritional supplement in an aqueous medium, wherein the water-soluble polysaccharide and water-soluble nutritional supplement are complexed with each other by ionic interactions in the spheroidal polysaccharide-stabilized complexes;
(ii) preparing an oil-supplement solution comprising at least one oil-soluble nutritional supplement dissolved in a food grade oil; and
(iii) blending the dispersion of spheroidal polysaccharide-stabilized complexes with the oil-supplement solution to produce the nutritional multi-supplement complex, wherein the nutritional multi-supplement complex comprises droplets of the oil-supplement solution encapsulated by the spheroidal polysaccharide-stabilized complexes.

2. The method of claim 1, further comprising: (iv) mixing the nutritional multi-supplement complex with a second polysaccharide to form a polysaccharide-encapsulated multi-supplement complex.

3. The method of claim 2, wherein the second polysaccharide comprises maltodextrin.

4. The method of claim 2, wherein the polysaccharide-encapsulated multi-supplement complex is spray-dried to form solid particles of the polysaccharide-encapsulated multi-supplement complex.

5. The method of claim 1, wherein the water-soluble polysaccharide in step (i) is selected from the group consisting of chitosan, carboxymethylcellulose, dextran, pectin, guar gum, xanthan gum, locust bean gum, gum arabic, and carrageenan.

6. The method of claim 1, wherein the water-soluble nutritional supplement comprises at least one water-soluble vitamin.

7. The method of claim 6, wherein the at least one water-soluble vitamin is selected from the group consisting of vitamin C and the B vitamins.

8. The method of claim 1, wherein the oil-soluble nutritional supplement comprises at least one oil-soluble vitamin.

9. The method of claim 8, wherein the at least one oil-soluble vitamin is selected from the group consisting of vitamin A, vitamin E, vitamin D, and vitamin K.

10. The method of claim 1, wherein blending in step (iii) comprises ultrasonication.

11. The method of claim 1, wherein step (i) is a three-step process comprising: (i-1) preparing a first aqueous solution containing at least one water-soluble nutritional supplement, (i-2) preparing a second aqueous solution containing the water-soluble polysaccharide, and (i-3) mixing the first and second aqueous solutions to form the spheroidal polysaccharide-stabilized complexes.

12. The method of claim 11, wherein the first and second aqueous solutions are adjusted to a pH of 2-8.

13. The method of claim 11, wherein the first and second aqueous solutions are adjusted to a pH of 4-6.

14. The method of claim 1, wherein the food grade oil is a vegetable oil.

15. The method of claim 1, wherein the at least one water-soluble nutritional supplement is present in an amount of 1 mg/mL to 100 mg/mL by volume of the aqueous medium, and the at least one oil-soluble nutritional supplement is present in an amount of 1 mg/mL to 100 mg/mL by volume of the food grade oil.

16. A nutritional multi-supplement complex comprising droplets of an oil-supplement solution encapsulated by spheroidal polysaccharide-stabilized complexes, wherein: (a) the oil-supplement solution comprises at least one oil-soluble nutritional supplement dissolved in a food grade oil, and (b) the spheroidal polysaccharide-stabilized complexes comprise a water-soluble polysaccharide and at least one water-soluble nutritional supplement in an aqueous medium, wherein the water-soluble polysaccharide and water-soluble nutritional supplement are complexed with each other by ionic interactions in the spheroidal polysaccharide-stabilized complexes.

17. The nutritional multi-supplement complex of claim 16, wherein the multi-supplement complex is encapsulated by a second polysaccharide.

18. The nutritional multi-supplement complex of claim 17, wherein the second polysaccharide comprises maltodextrin.

19. The nutritional multi-supplement complex of claim 17, wherein the multi-supplement complex is in solid (spray-dried) particulate form.

20. The nutritional multi-supplement complex of claim 16, wherein the water-soluble polysaccharide is selected from the group consisting of chitosan, carboxymethylcellulose, dextran, pectin, guar gum, xanthan gum, locust bean gum, gum arabic, and carrageenan.

21. The nutritional multi-supplement complex of claim 16, wherein the water-soluble nutritional supplement comprises at least one water-soluble vitamin.

22. The nutritional multi-supplement complex of claim 21, wherein the at least one water-soluble vitamin is selected from the group consisting of vitamin C and the B vitamins.

23. The nutritional multi-supplement complex of claim 16, wherein the oil-soluble nutritional supplement comprises at least one oil-soluble vitamin.

24. The nutritional multi-supplement complex of claim 23, wherein the at least one oil-soluble vitamin is selected from the group consisting of vitamin A, vitamin E, vitamin D, and vitamin K.

25. The nutritional multi-supplement complex of claim 16, wherein the food grade oil is a vegetable oil.

26. A beverage comprising the nutritional multi-supplement complex according to claim 16 homogeneously dispersed in a base aqueous medium suitable for human consumption.

27. The beverage of claim 26, further comprising one or more components from a tea plant.

28. A tea bag comprising the nutritional multi-supplement complex according to claim 16 contained within a tea bag enclosure.

29. The tea bag of claim 28, further comprising one or more components from a botanical source.

30. The tea bag of claim 28, further comprising one or more components from a tea plant.

31. The tea bag of claim 28, wherein the tea bag has a pore size cut-off sufficient to retain the complex in the tea bag.
